(19) United States
(12) Patent Application Publication
(10) Pub. No.: US 2024/0116051 A1
(43) Pub. Date: Apr. 11, 2024

(54) Device and Method for Separating Blood Plasma from Whole Blood

(71) Applicant: Robert Bosch GmbH, Stuttgart (DE)

(72) Inventor: Hannah Bott, Straubenhardt (DE)

(21) Appl. No.: 18/263,819

(22) PCT Filed: Feb. 8, 2022
(86) PCT No.: PCT/EP2022/052928

§ 371 Date: Aug. 1, 2023

(30) Foreign Application Priority Data
Feb. 8, 2021 (DE) 10 2021 201 146.2

(51) Int. Cl. B01L 3/00

(52) U.S. Cl. CPC B01L 3/502753; B01L 2200/0652

(57) ABSTRACT

A microfluidic device includes at least one separation chamber having an inlet and an outlet. The inlet opens into the separation chamber at a lower height than the outlet. In order to separate blood plasma from whole blood, whole blood is introduced into the separation chamber of the microfluidic device, blood cells are sedimented from the whole blood, and blood plasma separated in the process is sublayered using a transport medium which has a higher density than the blood plasma.

The present invention relates to a microfluidic device. Furthermore, the present invention relates to a method for separating blood plasma from whole blood using the microfluidic device.

### PRIOR ART

In many in vitro diagnostic tests, blood serves as the starting sample material. For example, to measure a specific DNA segment or protein, the blood is processed. For many diagnostics, the analysis of blood plasma components is of interest.

For in vitro diagnostics (IVD), which are measured on the patient without sending the sample to a central laboratory, sample preparation is performed by a person close to the patient. Since the latter usually does not have experienced laboratory process handling knowledge, the number of fluidic steps must be reduced to a minimum of simple manual steps. This can be realized using microfluidic lab-on-chip devices. These can be pneumatically based and can integrate multiple fluidic process and measurement steps. However, in such pneumatically operated, microfluidic devices, centrifugation steps are generally not possible, which are used as standard in everyday laboratory work to obtain blood plasma.

DE 10 2018 216 308 A1 describes a microfluidic system in which particles can be sedimented from a fluid volume. This can be used to sediment blood cells from serum. Sedimentation takes place in a chamber into which several channels open and which may be divided into separate zones.

### DISCLOSURE OF THE INVENTION

The microfluidic device comprising at least one separation chamber having an inlet and an outlet. The inlet opens into the separation chamber at a lower height than the outlet. In other words, in particular when the device is used as intended, the outlet is located higher in relation to gravity than the inlet in the chamber. The device has the advantage that, due to the greater height of the outlet, a first phase of a liquid present in the separation chamber can easily be partially discharged via the outlet from separated phase present below the first phase.

This device can preferably be used for automatable and quantifiable sedimentation-based separation of whole blood into blood plasma and cellular components. The different heights of the inlet and outlet have the advantage that, after sedimentation of the blood, blood plasma can be discharged separately from the rest of the blood via the outlet from the separation chamber, preferably by displacement of the blood plasma by means of a transport medium supplied through the inlet. Thus, advantageously, the blood plasma from the outlet can be removed from the separation chamber while the cellular components remain in the separation chamber.

In order to monitor such a transport process, it is preferred that at least one sensor is arranged in the separation chamber. The sensor is particularly preferably an optical sensor, most preferably a brightness sensor or a camera. By means of such a sensor, it is possible to monitor when a phase boundary between the blood plasma and residual whole blood, which is now enriched with the sedimented cellular components and may also be referred to as residual blood, moves into the pick-up area of the sensor, thus ensuring that as much blood plasma as possible but no residual whole blood or cellular components enter the outlet.

To ensure that no cellular components enter the outlet, it is further preferred that a filter is arranged at and/or in the outlet. Although blood plasma can pass through such a filter, cellular components of the blood are retained. However, due to the high content of cellular components in whole blood, the filter-based approach is not suitable for separating a large amount of whole blood, as the filter would clog. In other words, the invention advantageously enables the combination of sedimentation-based phase separation of the blood plasma and use of the filter to obtain blood plasma that is as particle-free as possible in large quantities without the filter clogging prematurely. The combination of sedimentation and filtration described here thus enables automated separation of blood plasma even for larger volumes of more than 50 μl, for example. The filter can preferably be a filter for separating blood plasma from blood, in particular a membrane filter suitable for this purpose, such as the Vivid™ Plasma Separation Membrane.

Basically, the inlet and outlet can be arranged in such a way that the inlet is located at the bottom of the separation chamber and the outlet is located at its top. Preferably, however, the outlet is located in a side wall of the separation chamber. This allows sample input to be arranged in the top side of the separation chamber. When the microfluidic device is designed as a lab-on-chip, the sample input can perform the function of a world-to-chip interface through which whole blood can be input directly into the separation chamber. It is then not necessary to first transport it through channels of the microfluidic device to then pass it through the inlet into the separation chamber.

Furthermore, it is preferred that the outlet opens into a further chamber. The additional chamber can, for example, have the same or approximately the same volume as the separation chamber. The further chamber further has a common wall with the separation chamber. The separation chamber and the further chamber can thus be designed as parts of a superordinate chamber, which is divided into the separation chamber and the further chamber by the common wall. The additional chamber enables the collection of blood plasma that has been displaced from the separation chamber. As no pipe is required between the outlet of the separation chamber and the further chamber, but the outlet of the separation chamber simultaneously functions as the inlet of the further chamber, a very space-saving arrangement of the two chambers is possible.

In order to increase the transfer efficiency from the separation chamber to the further chamber, it is further preferred that the separation chamber and the further chamber are inclined together in the direction of the further chamber, in particular inclined with respect to an underside of the device The inclination is preferably at least 20°. When the device is used as intended, such an advantageous inclination facilitates gravity-driven transport of blood plasma into the further chamber after a transport medium has been introduced into the separation chamber. According to a preferred embodiment, a bottom of the separation chamber may be inclined with respect to a bottom side of the device. In other words, a flat bottom surface of the separation chamber is not parallel to a flat bottom surface of the device, but is arranged at an opening angle to each other, for example an opening angle between 5 and 30 degrees, preferably between 10 and 25 degrees, most preferably between 15 and 25 degrees.

A microfluidic pumping device and valves can be implemented, for example, by pneumatically actuating deflection of a polymer membrane into recesses in a polymer substrate, which further contains microfluidic channels and the separation chamber. Suitable materials of the separation chamber are in particular polymers such as polycarbonate (PC), polypropylene (PP), polyethylene (PE), polymethyl methacrylate (PMMA), polydimethylsiloxane (PDMS) or thermoplastic elastomers (TPE). In particular, polyurethane (TPU) or styrene block copolymers (TPS) can be used as thermoplastic elastomers. The processing of such polymers to form the separation chamber can be carried out in particular by high-throughput processes such as injection molding, thermoforming, stamping or laser transmission welding.

The volume of the separation chamber is preferably in the range of 20 μl to 1000 μl and particularly preferably in the range of 50 μl to 100 μl.

In the method for separating blood plasma from whole blood, the first step is to introduce the whole blood into the separation chamber of the microfluidic device. If the microfluidic device has a sample input, the insertion can be performed through this sample input. Otherwise, the whole blood is passed through the inlet into the separation chamber.

After the whole blood has entered the separation chamber, sedimentation of blood cells from the whole blood takes place there. Sedimentation can basically be gravity-driven by leaving the whole blood in the separation chamber for a predetermined period of time. To accelerate sedimentation, however, it may be preferable to add magnetic beads to the whole blood and then enhance sedimentation of the blood cells by applying an external magnetic field.

After sedimentation of the blood cells from the whole blood is complete, discharge of at least a portion of blood plasma occurs via the outlet of the device, with the blood plasma being present as a separate phase above the residual blood due to sedimentation. According to a preferred embodiment of the method, the discharge can be performed by sublayering with a transport medium. The transport medium preferably has a higher density than the blood plasma. Suitable transport media, which have a higher density than blood plasma and are also immiscible with it, are in particular fluorinated hydrocarbons. Particularly suitable transport media are selected from the group consisting of bis(nonafluorobutyl)(trifluoromethyl)amine (FC40), perfluorotripentylamine (FC70), 3-ethoxy-1,1,1,2,3,4,4,5,5,6,6-dodecafluoro-2-(trifluoromethyl)-hexane (HFE7500) and 1,1,1,2,2,3,4,5,5,5-decafluoro-methoxy-4-(trifluoromethyl)-pentane (HFE7300). Alternatively, the transport medium can also be further blood or another liquid, which is introduced into the separation chamber through the inlet.

Sublayering transports the blood plasma in the direction of the outlet through the separation chamber and finally displaces it through the outlet. Residual whole blood, in which the sedimented blood cells have collected, forms a phase separate from the blood plasma in the separation chamber, which can also be transported through the separation chamber during sublayering with the transport medium.

In one embodiment of the method, to prevent residual whole blood from entering the effluent, it is preferred that sublayering continues until a phase boundary between the blood plasma and the residual whole blood reaches a predeterminable level in the separation chamber. This can be detected in particular by means of a sensor in the separation chamber, as also described above.

In another embodiment of the method, sublayering continues until a predeterminable amount of the transport medium has been introduced into the separation chamber. This predeterminable quantity is selected in particular as a function of the volume of the separation chamber, the height at which the outlet is arranged, and the volume of whole blood introduced into the separation chamber. No sensor is required for this embodiment of the method. Furthermore, the transferred volume of blood plasma is known by the given amount of transport medium and can be included as a parameter for subsequent analyses.

The remainder of the whole blood is then transported out of the separation chamber, preferably by means of the transport medium, to prepare it for reuse. In one embodiment of the method, this may involve displacing the remainder of the whole blood through the outlet as well by introducing further transport medium into the separation chamber after the blood plasma has been further transported to its destination in the microfluidic device. The whole blood passed through the outlet can then be transported, for example by means of a valve, into another line and used for further analysis of the cellular components.

In another embodiment of the method, the transport medium is provided to be pumped back through the inlet of the separation chamber. The remaining whole blood resting on the transport medium follows the transport medium and also leaves the separation chamber through the inlet.

### BRIEF DESCRIPTION OF THE DRAWINGS

Exemplary embodiments of the invention are illustrated in the drawings and are explained in more detail in the following description.

FIG. 1 shows a schematic sectional view of a separation chamber of a microfluidic device according to an embodiment of the invention.

FIGS. 2a to 2d show steps of a method according to an embodiment of the invention, which take place in the separation chamber according to FIG. 1.

FIGS. 3a to 3c show flowcharts of different embodiments of the method according to the invention.

FIG. 4 shows a sectional view of a separation chamber in another embodiment of the microfluidic device according to the invention.

FIG. 5 shows a sectional view of a separation chamber in still another embodiment of the microfluidic device according to the invention.

FIGS. 6a to 6e show steps of a method according to an embodiment of the invention, which takes place in the separation chamber according to FIG. 5.

FIG. 7 shows a sectional view of a separation chamber in a microfluidic device according to still another embodiment of the invention.

FIG. 8 shows a sectional view of a separation chamber in a microfluidic device according to still another embodiment of the invention.

### EXEMPLARY EMBODIMENTS OF THE INVENTION

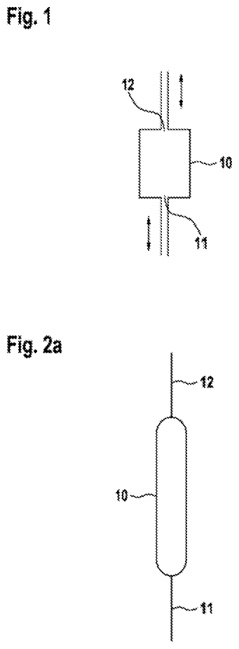

In a first embodiment of the invention, a microfluidic device for analyzing blood samples comprises a separation chamber 10 shown in FIG. 1. The volume of the separation chamber 10 in the present embodiment is 75 μl. It has an inlet 11 at its bottom and an outlet 12 at its top. A transport of fluids through the inlet 11 and the outlet 12 occurs in the microfluidic device by the pneumatically actuated deflection of a polymer membrane into recesses in a polymer substrate.

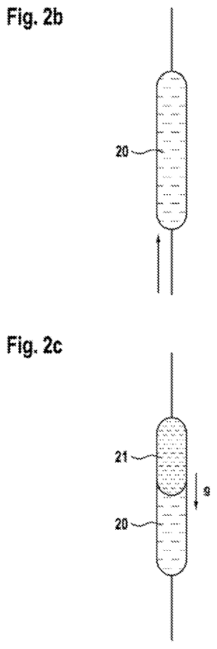

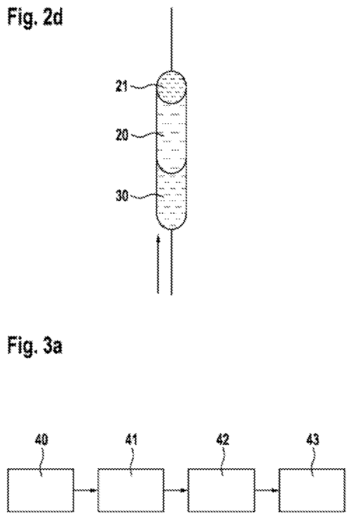

FIGS. 2a to 2d show how, in a first embodiment of the method according to the invention, blood plasma is separated from whole blood in the separation chamber 10 according to the first embodiment of the microfluidic device. As shown in FIG. 2a, the separation chamber 10 is initially empty. Whole blood 20 is then introduced through the inlet 11 into the separation chamber 10 until it is completely filled with whole blood 20, as shown in FIG. 2b. FIG. 2c shows that after some time a gravitationally g driven sedimentation of blood cells from the whole blood 20 occurs, so that a phase of blood plasma 21 forms above the whole blood 20. FIG. 2d shows how, finally, a transport medium 30, which in the present embodiment is FC40, is introduced into the separation chamber 10 through the inlet 11 to sublayer the whole blood 20 and blood plasma 21 with it. In this process, the blood plasma 21 is gradually displaced from the separation chamber 10 by the outlet 12. After a predetermined volume of the transport medium 30 has been introduced into the separation chamber 10, the sublayering is terminated.

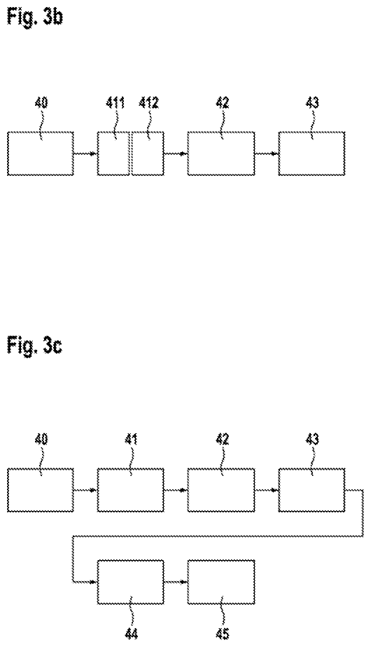

The sequence of this method is shown in FIG. 3a. The introduction 40 of the whole blood 20 into the separation chamber 10 is followed first by sedimentation 41 of blood cells from the whole blood 20 and then by sublayering 42 of the blood plasma and the remaining whole blood 20 with the transport medium 30. After the blood plasma 21 has entered the outlet 12 in this manner, it is directed into another part of the microfluidic device by pumping operations in a final step 43.

FIG. 3b shows a modification of the method sequence according to FIG. 3a in a second embodiment of the method according to the invention. Sedimentation 41 is divided into two substeps 411, 412. In step 411, magnetic beads are introduced into the whole blood 20 through the inlet 11 and bind to the blood cells. To this end, the present embodiment provides that the magnetic beads comprise CD45 antibodies that bind to leukocytes. In the following step 412, an electromagnet located below the separation chamber 10 is turned on to initiate accelerated sedimentation of the blood cells.

A third embodiment of the method according to the invention for separating the blood plasma 21 from the whole blood 20 is shown in FIG. 3c. The method steps 40 to 43 already described, in which method step 41 can be replaced by substeps 411 and 412 if necessary, are followed by transporting 44 the remaining whole blood 20 out of the separation chamber 10. This can optionally be done either by directing additional transport medium 30 into the separation chamber 10 through the inlet 11 to displace the remaining whole blood 20 from the separation chamber 10 through the outlet 12, or by pumping the transport medium 30 back so that it exits the separation chamber 10 through the inlet 11, taking the remaining whole blood 20 with it. This is followed by transfer 45 of the remaining whole blood 20 by pumping to another part of the microfluidic device.

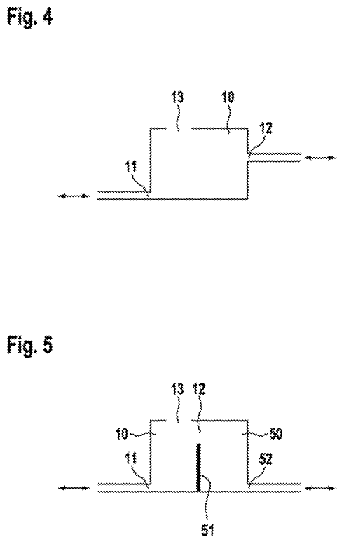

FIG. 4 shows the design of the separation chamber 10 in a second embodiment of the microfluidic device. Here, the inlet 11 and the outlet 12 are not located on the bottom and the top of the separation chamber 10. Instead, the inlet 11 opens into the lower end of a side wall of the separation chamber 10. The outlet 12 ends in a side wall of the separation chamber 10 above the inlet 11. A sample entry 13 in the form of an opening is provided in the top of the separation chamber 10, which connects the separation chamber 10 to an interface region not shown, which in the present embodiment has a volume of 1 ml. Through this interface region, whole blood 20 can be introduced directly into the separation chamber 10 without a detour via the inlet 11.

FIG. 5 shows a separation chamber 10 in a third embodiment of the microfluidic device. A superior chamber is divided by a common wall 51 into the separation chamber 10 and another chamber 50. While the inlet 11 is designed in the same manner as in the second embodiment of the microfluidic device, the outlet 12 is a free-standing area above the common wall 51 connecting the separation chamber 10 to the further chamber 50. The further chamber 50 has a further outlet 52 on the underside of one of its side walls. Also in this embodiment of the microfluidic device, a sample inlet 13 is provided which allows direct access to the separation chamber 10 through the top of the chamber.

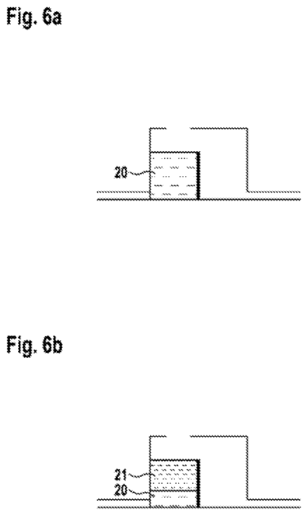

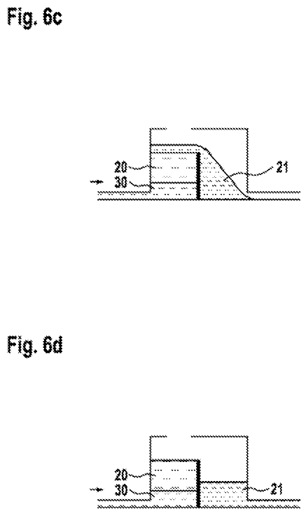

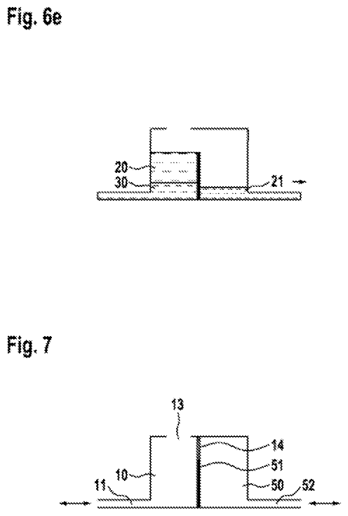

FIGS. 6a through 6e show the sequence of a fourth embodiment of the method according to the invention using the microfluidic device according to the third embodiment. As shown in FIG. 6a, the separation chamber 10 is initially filled with whole blood 20 up to the upper edge of the common wall 51. Sedimentation then takes place, as shown in FIG. 6b, which causes a phase of blood plasma 21 to settle above the whole blood 20. Now, as shown in FIG. 6c, when a transport medium 30 is introduced into the separation chamber 10 through the inlet 11, the liquid level in the separation chamber 10 rises to the point where the blood plasma 21 spills over into the further chamber 50. Introduction of the transport medium 30 is terminated after introduction of a volume at which the upper edge of the phase of whole blood 20 is still just below the upper edge of the common wall 51, as expected. This achieves the separation of whole blood 20 in the separation chamber 10 and blood plasma 21 in the further chamber 50 shown in FIG. 6d. FIG. 6e shows how finally the blood plasma 21 is pumped out of the further chamber 50 through the outlet 12.

FIG. 7 shows how the separation chamber 10 is configured in a fourth embodiment of the microfluidic device. In this respect, it is largely similar to the separation chamber 10 according to the third embodiment of the invention. However, a filter 14 is arranged in the outlet 12 to prevent blood cells from passing from the separation chamber 10 to the further chamber 50. In this way, it is possible to introduce a larger volume of transport medium 30 into the separation chamber 10 without the risk of transferring whole blood 20 into the other chamber 50. However, since the transfer of the whole blood 20 is prevented by the filter 14 even after the blood plasma is removed from the further chamber 50, in this embodiment of the microfluidic device it is not possible to remove the remaining whole blood 20 from the separation chamber 10 by introducing further transport medium 30, but instead it must be aspirated through the inlet 11 together with the transport medium 30. The filter may preferably be a blood plasma filter 14 for separating blood plasma from blood, in particular a membrane filter suitable for this purpose, such as the Vivid™ Plasma Separation Membrane. For example, the filter 14 may have an area between 25 and 400 square millimeters, such as 100 square millimeters.

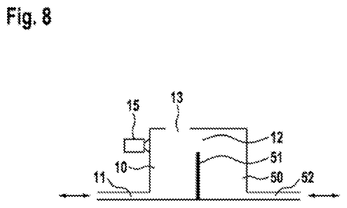

Finally, FIG. 8 illustrates an embodiment of a separation chamber 10 in a fifth embodiment of the microfluidic device. This is also a minor modification of the separation chamber 10 according to the third embodiment of the microfluidic device. Instead of the filter 14 or alternatively in addition to the filter 14, a sensor 15 in the form of a brightness sensor is arranged in the separation chamber 10. This is positioned to capture the top edge of the common wall 51. Instead of introducing a predetermined amount of the transport medium 30 into the separation chamber 10, this embodiment of the microfluidic device provides that the introduction of the transport medium 30 continues until the sensor 15 detects that the phase boundary between blood plasma 21 and whole blood 20 has reached the upper edge of the common wall 51.

1. A microfluidic device comprising at least one separation chamber with an inlet and an outlet, wherein the inlet is configured to open into the separation chamber at a lower height than the outlet.

2. The microfluidic device according to claim 1, wherein at least one sensor is arranged in the separation chamber.

3. The microfluidic device according to claim 1, further comprising a filter arranged at and/or in the outlet.

4. The microfluidic device according to claim 1, wherein the outlet is disposed in a side wall of the separation chamber and a sample inlet is disposed in a top of the separation chamber.

5. The microfluidic device according to claim 4, wherein the outlet is configured to open into another chamber having a common wall with the separation chamber.

6. The microfluidic device according to claim 5, wherein the separation chamber and the further chamber are inclined together towards the further chamber.

7. A method for separating blood plasma from whole blood, comprising:
introducing the whole blood into the separation chamber of a microfluidic device according to claim 1,
sedimenting blood cells from the whole blood, and
at least partially discharging blood plasma separated by sedimentation via the outlet.

8. The method according to claim 7, wherein the step of at least partially discharging blood plasma is performed by sublayering with a transport medium.

9. The method according to claim 8, wherein the step of sublayering is continued until a phase boundary between the blood plasma and a residue of the whole blood has reached a predeterminable height in the separation chamber.

10. The method according to 8, wherein the step of sublayering is continued until a predeterminable amount of the transport medium has been introduced into the separation chamber.

11. The method according to claim 7, wherein a remainder of the whole blood is transported out of the separation chamber by way of a transport medium.

12. The method according to claim 7, wherein the step of at least partially discharging blood plasma is performed by sublayering with a transport medium which has a higher density than the blood plasma.
